(19) United States
(12) Patent Application Publication
(10) Pub. No.: US 2008/0064940 A1
(43) Pub. Date: Mar. 13, 2008

(54) Sensor cable design for use with spectrophotometric sensors and method of using the same

(76) Inventor: William B. Raridan, Pleasanton, CA (US)

Correspondence Address: Covidien;IP Counsel - Respiratory & Monitoring Solutions, 60 Middletown Avenue, North Haven, CT, 06473, US

(21) Appl. No.: 11/519,714

(22) Filed: Sep. 12, 2006

(51) Int. Cl. A61B 5/1455

(52) U.S. Cl. 600/344; 600/310; 600/323

(57) ABSTRACT

A sensor cable is provided that includes a sensor point of attachment configured to attach to a spectrophotometric sensor, and a connector configured to attach to a patient monitor. The sensor cable includes a coiled portion between the sensor point of attachment and the monitor connector. A sensor assembly and system including such a cable are also provided.

### BACKGROUND OF THE INVENTION

1. Field Of The Invention

The present invention relates generally to medical devices and, more particularly, to sensor cables used for connecting a sensor to a monitor.

2. Description Of The Related Art

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient.

Pulse oximeters typically utilize a non-invasive sensor that is placed on or against a patient's tissue that is well perfused with blood, such as a patient's finger, toe, forehead or earlobe. The pulse oximeter sensor emits light and photoelectrically senses the absorption and/or scattering of the light after passage through the perfused tissue. The data collected by the sensor may then be used to calculate one or more of the above physiological characteristics based upon the absorption or scattering of the light. More specifically, the emitted light is typically selected to be of one or more wavelengths that are absorbed or scattered in an amount related to the presence of oxygenated versus de-oxygenated hemoglobin in the blood. The amount of light absorbed and/or scattered may then be used to estimate the amount of the oxygen in the tissue using various algorithms.

Additionally, a sensor cable is typically employed to connect the sensor on the patient to the monitoring equipment. Such sensor cables, once connected to the patient and monitor, may be damaged by movements of the patient or monitoring equipment. For example, the sensor cable may be inflexible and unable to prevent damage to the cable or the connections to the sensor or monitor when exposed to longitudinal and/or rotational forces when the patient moves. Furthermore, transmitted noise may interfere with the signal transmission from the sensor to the monitor along the cable.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

There is provided a sensor assembly that includes: a sensor body of a spectrophotometric sensor; and a sensor cable comprising a coiled portion. In addition, there is also provided a system that includes: a monitor; and the sensor assembly described above.

There is also provided a method of connecting a sensor assembly to a monitor that includes: placing a sensor on a location on the body of a patient; and connecting the sensor assembly to a monitor using a sensor cable comprising a coiled portion.

There is also provided a sensor cable that includes: a point of attachment configured to attach to a spectrophotometric sensor; a connector configured to attach to a patient monitor; and a coiled portion disposed between the point of attachment and the connecter.

### BRIEF DESCRIPTION OF THE DRAWINGS

Advantages of the invention may become apparent upon reading the following detailed description and upon reference to the drawings in which:

FIG. 1 illustrates a patient monitoring system including a sensor cable having a coiled portion and a sensor, in accordance with aspects of the present technique; and

FIG. 2 depicts a sensor cable for use with the system of FIG. 1, in accordance with aspects of the present technique.

### DETAILED DESCRIPTION OF SPECIFIC EMBODIMENTS

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

It is desirable to provide a comfortable and conformable reusable patient sensor, such as for use in pulse oximetry or other applications utilizing spectrophotometry, In accordance with some aspects of the present technique, a reusable patient sensor is provided that is attached to or attachable to a sensor cable that allows for greater patient movement while preventing the deleterious effects of such movement. For example, in one embodiment, a spectrophotometric sensor is provided that includes a sensor cable having a coiled portion.

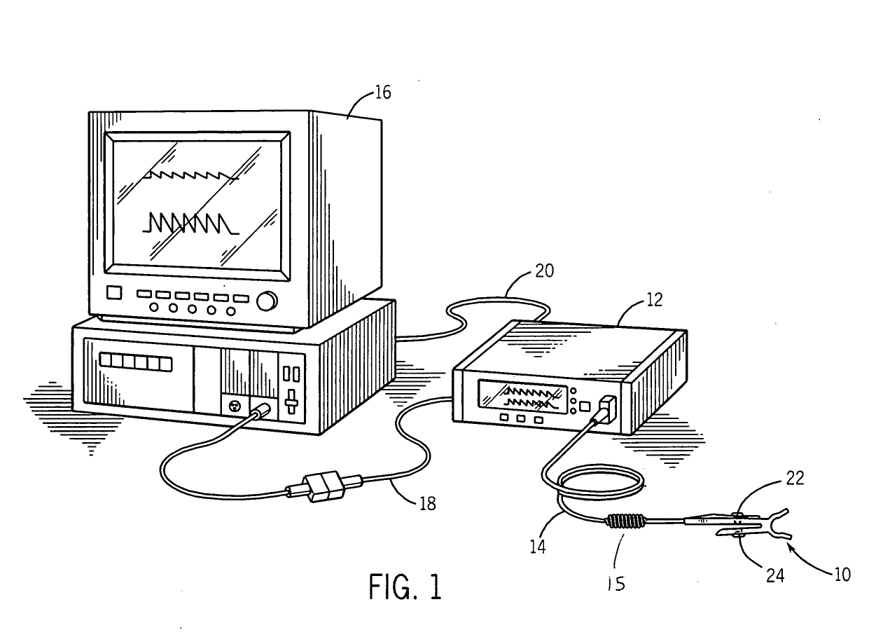

Prior to discussing such exemplary sensors and sensor cables in detail, it should be appreciated that such sensors are typically designed for use with a patient monitoring system. For example, referring now to FIG. 1, a sensor 10 according to the present invention may be used in conjunction with a patient monitor 12. In the depicted embodiment, a sensor cable 14 connects the sensor 10 to the patient monitor 12. As will be appreciated by those of ordinary skill in the art, the sensor 10 and/or the sensor cable 14 may include or incorporate one or more integrated circuit devices or electrical devices, such as a memory, processor chip, or resistor, that may facilitate or enhance communication between the sensor 10 and the patient monitor 12. Likewise the sensor cable 14 may be an adaptor cable, with or without an integrated circuit or electrical device, for facilitating communication between the sensor 10 and various types of monitors, including older or newer versions of the patient monitor 12 or other physiological monitors. As will be appreciated by those of ordinary skill in the art, the sensor cable 14 are typically used to transmit control or timing signals from the monitor 12 to the sensor 10 and/or to transmit acquired data from the sensor 10 to the monitor 12.

In one embodiment, the patient monitor 12 may be a suitable pulse oximeter, such as those available from Nellcor Puritan Bennett Inc. In other embodiments, the patient monitor 12 may be a monitor suitable for measuring tissue water fractions, or other body fluid related metrics, using spectrophotometric or other techniques. Furthermore, the monitor 12 may be a multi-purpose monitor suitable for performing pulse oximetry and measurement of tissue water fraction, or other combinations of physiological and/or biochemical monitoring processes, using data acquired via the sensor 10. Furthermore, to upgrade conventional monitoring functions provided by the monitor 12 to provide additional functions, the patient monitor 12 may be coupled to a multi-parameter patient monitor 16 via a cable 18 connected to a sensor input port and/or via a cable 20 connected to a digital communication port.

The sensor 10, in the example depicted in FIG. 1, is a clip-style sensor that is overmolded to provide a unitary or enclosed assembly. The sensor 10 includes an emitter 22 and a detector 24 which may be of any type suitable for spectrophotometric measurement. For example, the emitter 22 may be one or more light emitting diodes adapted to transmit one or more wavelengths of light, such as in the red to infrared range, and the detector 24 may be a photodetector, such as a silicon photodiode package, selected to receive light in the range emitted from the emitter 22. In the depicted embodiment, the sensor 10 is coupled to a sensor cable 14 that is responsible for transmitting electrical and/or optical signals to and from the emitter 22 and detector 24 of the sensor 10. The sensor cable 14 may be permanently coupled to the sensor 10, or it may be removably coupled to the sensor 10—the latter alternative being more useful and cost efficient in situations where the sensor 10 is disposable.

The sensor 10 described above is generally configured for use as a “transmission type” sensor for use in spectrophotometric applications, though in some embodiments it may instead be configured for use as a “reflectance type sensor.” Transmission type sensors include an emitter and detector that are typically placed on opposing sides of the sensor site. If the sensor site is a fingertip, for example, the sensor 10 is positioned over the patient's fingertip such that the emitter and detector lie on either side of the patient's nail bed. For example, the sensor 10 is positioned so that the emitter is located on the patient's fingernail and the detector is located opposite the emitter on the patient's finger pad. During operation, the emitter shines one or more wavelengths of light through the patient's fingertip, or other tissue, and the light received by the detector is processed to determine various physiological characteristics of the patient.

Reflectance type sensors generally operate under the same general principles as transmittance type sensors. However, reflectance type sensors include an emitter and detector that are typically placed on the same side of the sensor site. For example, a reflectance type sensor may be placed on a patient's fingertip such that the emitter and detector are positioned side-by-side. Reflectance type sensors detect light photons that are scattered back to the detector.

For pulse oximetry applications using either transmission or reflectance type sensors the oxygen saturation of the patient's arterial blood may be determined using two or more wavelengths of light, most commonly red and near infrared wavelengths. Similarly, in other applications a tissue water fraction (or other body fluid related metric) or a concentration of one or more biochemical components in an aqueous environment may be measured using two or more wavelengths of light, most commonly near infrared wavelengths between about 1,000 nm to about 2,500 nm. It should be understood that, as used herein, the term “light” may refer to one or more of infrared, visible, ultraviolet, or even X-ray electromagnetic radiation, and may also include any wavelength within the infrared, visible, ultraviolet, or X-ray spectra.

As noted above, the overmolded sensor 10 discussed herein may be configured for either transmission or reflectance type sensing. Furthermore, the sensor 10 may include various structural and functional features designed to facilitate its use. An example of such a sensor and its use and construction may be found in U.S. application Ser. No. 11/199,524 titled “Medical Sensor and Technique for Using the Same” and filed on Aug. 8, 2005, which is hereby incorporated by reference in its entirety. As will be appreciated by those of ordinary skill in the art, however, such discussion is merely exemplary and is not intended to limit the scope of the present technique.

In the embodiment depicted in FIG. 1, the sensor 10 is depicted as being connected to the patient monitor 12 by the sensor cable 14. In one embodiment, the sensor cable 14 includes a series of helical coils, i.e., a coiled portion 15, along some or all of the length of the cable. The coiled portion 15 allows the cable 14 to be placed in tension and/or rotated without damage to the cable itself and without transmitting the tension or rotation throughout the length of the sensor cable 14. The flexibility of the sensor cable 14 provided by the coiled portion 15 allows for easier draping and routing of the cable and provides for an increase in the inductance of the cable compared to a comparable straight cable.

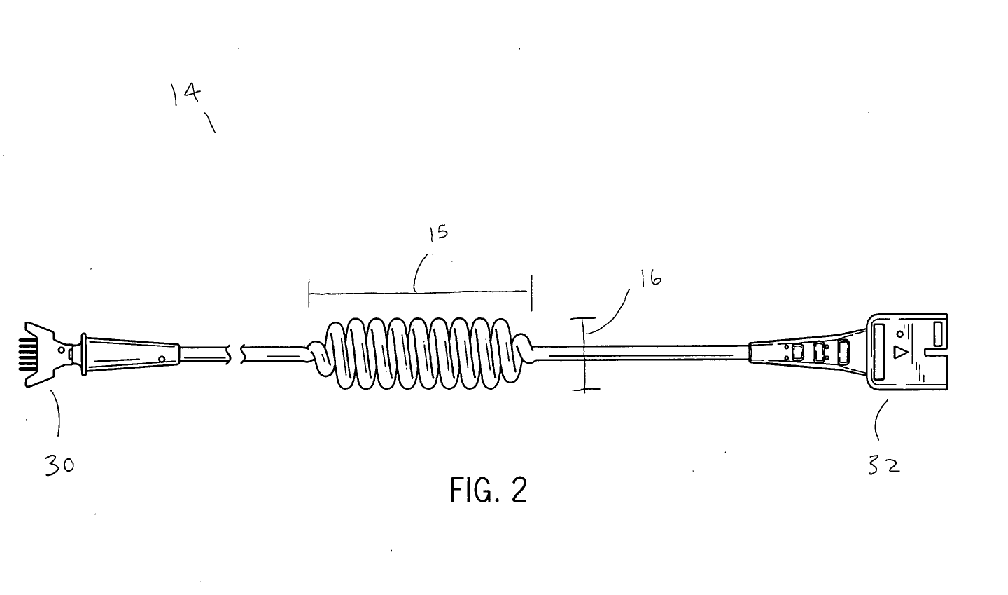

Referring now to FIG. 2, the sensor cable 14 is depicted in greater detail, including a sensor point of attachment 30 and a monitor connector 32. The depicted exemplary sensor cable includes a coiled portion 15, consisting of 10 coils±0.5 coils. In the exemplary embodiment depicted in FIG. 2, the sensor cable 14 has a length of approximately 11 feet (3.35 m), and the length of the coiled portion 15 is approximately 2±0.5 inches (5.08 cm±1.27 cm). Alternative embodiments may vary the number of coils, the length of the sensor cable 14, or the length of the coiled portion 15. The ratio of the length of the coiled portion 15 respective to the length of the sensor cable 14 may also vary, including embodiments in which the length of the coiled portion may be one-quarter, one-half, or all of the length of the sensor cable. Additionally, the embodiment in FIG. 2 includes a coil diameter 16 of approximately 0.74±0.30 inches (1.88 cm±0.76 cm), which may vary in other embodiments. As compared to a straight cable, the coiled portion 15 of the sensor cable 14 results in an increased inductance. As will be appreciated by those skilled in the art, such an increase in inductance reduces transmitted noise and enhances the signal to noise ratio. Such inductance may be adjusted by increasing or decreasing the number of coils or by adjusting the relative geometry of the coiled portion 15, i.e. length, diameter, and so forth.

Further, as will be appreciated by those of ordinary skill in the art, the coiled portion of the sensor cable 14 provides various benefits. For example, the coiled portion 15, as discussed above, provides a mechanism for absorbing or reducing rotational torque and/or longitudinal tension caused by the rotational or translational movement of one end of the sensor cable 14 relative to the other. Likewise, the enhanced flexibility provided by the coiled portion 15 helps prevent damage to internal and or attached components of the sensor cable 14 as well as facilitating the routing of the sensor cable 14.

While the exemplary medical sensors 10 discussed herein are some examples of medical devices incorporating a sensor cable as described herein, other such devices are also contemplated and fall within the scope of the present disclosure. For example, other medical sensors and/or contacts applied externally to a patient may be advantageously applied using a sensor body and sensor cable as discussed herein. Similarly, and as noted above, devices for measuring tissue water fraction or other body fluid related metrics may utilize a sensor and sensor cable as described herein. Likewise, other spectrophotometric applications where a probe is attached to a patient may utilize a sensor and sensor cable as described herein.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims. Indeed, the present techniques may not only be applied to transmission type sensors for use in pulse oximetry, but also to retroflective and other sensor designs as well. Likewise, the present techniques are not limited to use on fingers and toes but may also be applied to placement on other body parts such as in embodiments configured for use on the ears or nose.

What is claimed is:

1. A sensor assembly, comprising:
a sensor body of a spectrophotometric sensor; and
a sensor cable comprising a coiled portion.

2. The sensor assembly of claim 1, wherein the sensor body comprises an overmolded coating.

3. The sensor assembly of claim 1, wherein the sensor body comprises one or more light emitting diodes and a photodetector.

4. The sensor assembly of claim 1, wherein the sensor assembly comprises at least one of a pulse oximetry sensor, a sensor for measuring a water fraction, or a combination thereof.

5. The sensor assembly of claim 1, wherein the coiled portion is approximately 2 inches long or greater.

6. The sensor assembly of claim 1, wherein the coiled portion is approximately one-quarter of the length of the sensor cable.

7. The sensor assembly of claim 1, wherein the coiled portion is approximately one-half of the length of the sensor cable.

8. The sensor assembly of claim 1, wherein the coiled portion is substantially all of the length of the sensor cable.

9. The sensor assembly of claim 1, wherein the coiled portion allows rotation of one end of the cable without rotational tension at the other end.

10. The sensor assembly of claim 1, wherein the coiled portion absorbs longitudinal tension to reduce longitudinal tension at one or both ends.

11. A monitoring system, comprising:
a monitor;
a sensor assembly adapted to be coupled to the monitor, the sensor assembly comprising:
a sensor body of a spectrophotometric sensor; and
a sensor cable comprising a coiled portion.

12. The sensor assembly of claim 11, wherein the sensor body comprises an overmolded coating.

13. The sensor assembly of claim 11, wherein the sensor body comprises one or more light emitting diodes and a photodetector

14. The sensor assembly of claim 1 1, wherein the sensor assembly comprises at least one of a pulse oximetry sensor, a sensor for measuring a water fraction, or a combination thereof.

15. A method of connecting a sensor assembly to a monitor, comprising:
placing a sensor assembly on a location on the body of a patient; and
connecting the sensor assembly to a monitor using a sensor cable comprising a coiled portion.

16. The method of 15, wherein the sensor assembly comprises a sensor body having an overmolded coating.

17. The sensor assembly of claim 15, wherein the sensor assembly comprises one or more light emitting diodes and a photodetector

18. The sensor assembly of claim 15, wherein the sensor assembly comprises at least one of a pulse oximetry sensor, a sensor for measuring a water fraction, or a combination thereof.

19. A sensor cable, comprising:
a point of attachment configured to attach to a spectrophotometric sensor;
a connector configured to attach to a patient monitor; and
a coiled portion disposed between the point of attachment and the connector.

20. The sensor assembly of claim 19, wherein the coiled portion is approximately 2 inches long or greater.

21. The sensor assembly of claim 19, wherein the coiled portion is approximately one-quarter of the length of the sensor cable.

22. The sensor assembly of claim 19, wherein the coiled portion is approximately one-half of the length of the sensor cable.

23. The sensor assembly of claim 19, wherein the coiled portion is substantially all of the length of the sensor cable.

24. The sensor assembly of claim 19, wherein the coiled portion allows rotation of one end of the cable without rotational tension at the other end.

25. The sensor assembly of claim 19, wherein the coiled portion absorbs longitudinal tension to reduce longitudinal tension at one or both ends.
